(19) United States
(12) Patent Application Publication
(10) Pub. No.: US 2024/0257943 A1
(43) Pub. Date: Aug. 1, 2024

(54) TRAINING METHOD OF BRAIN ACTIVITY STATE CLASSIFICATION MODEL, BRAIN ACTIVITY STATE CLASSIFICATION METHOD, DEVICE, AND STORAGE MEDIUM

(71) Applicant: RUIANXING MEDICAL TECHNOLOGY(SUZHOU)CO.LTD, Jiangsu (CN)

(72) Inventors: Buwei YU, Jiangsu (CN); Tielin ZHANG, Jiangsu (CN); Han LU, Jiangsu (CN); Hongxing LIU, Jiangsu (CN)

(21) Appl. No.: 18/425,118

(22) Filed: Jan. 29, 2024

(30) Foreign Application Priority Data
Feb. 1, 2023 (CN) 202310073229.7

(51) Int. Cl. G16H 20/70; A61B 5/00; A61B 5/374; G16H 50/20

(52) U.S. Cl. CPC G16H 20/70; A61B 5/374; A61B 5/7267; G16H 50/20

(57) ABSTRACT

Provided are a training method of a brain activity state classification model, a brain activity state classification method, a device, and a storage medium. The training method includes acquiring pulse sequences of electroencephalography signal samples corresponding to multiple training tasks; and inputting the pulse sequences of the electroencephalography signal samples into an initial brain activity state classification model and training the brain activity state classification model based on a target rule. In a forward propagation stage in the target rule, Hebbian information corresponding to each synapse in the brain activity state classification model is updated according to the pulse sequences corresponding to the multiple training tasks; and in a backward propagation stage in the target rule, a weight of each synapse in the brain activity state classification model is determined according to the Hebbian information corresponding to each synapse and a backward propagation result.

### CROSS-REFERENCE TO RELATED APPLICATIONS

This application claims priority to Chinese patent application No. 202310073229.7 filed with the China National Intellectual Property Administration (CNIPA) on Feb. 1, 2023, the disclosure of which is incorporated herein by reference in its entirety.

### TECHNICAL FIELD

The present disclosure relates to the technical field of medical signal processing and, in particular, to a training method of a brain activity state classification model, a brain activity state classification method, a device, and a storage medium.

### BACKGROUND

In the past few decades, research on artificial intelligence has made rapid progress, especially on connectionist artificial neural network models, and great success has been achieved in tasks such as image recognition, target detection, speech recognition, and natural language processing. Optionally, the artificial neural network model may be used in clinical medical application scenarios. Through electroencephalography signal monitoring, signals from different brain areas can be classified, assisting the doctor in determining the source of a signal and confirming the brain state and physical condition of the patient for more accurate treatment.

In the related art, the artificial neural network is used for classifying brain activity states. However, in a scenario where data distribution continues to change, the artificial neural network model is plagued by the same catastrophic forgetting issue as traditional methods, that is, the learning of new knowledge may interfere with the memory of old knowledge, causing the brain activity state classification result to be less accurate and less efficient.

### SUMMARY

Embodiments of the present disclosure provide a training method and apparatus of a brain activity state classification model, a brain activity state classification method, a device, and a storage medium.

The embodiments of the present disclosure provide the technical schemes described below.

An embodiment of the present disclosure provides a training method of a brain activity state classification model. The method includes the steps described below.

Pulse sequences of electroencephalography signal samples corresponding to multiple training tasks for brain activity state classification are acquired.

The pulse sequences of the electroencephalography signal samples corresponding to the multiple training tasks are inputted into an initial brain activity state classification model, and the brain activity state classification model is trained based on a target rule. The step in which the brain activity state classification model is trained based on the target rule comprises the following: in a forward propagation stage in the target rule, Hebbian information corresponding to each synapse in the brain activity state classification model is updated according to the pulse sequences corresponding to the multiple training tasks; and in a backward propagation stage in the target rule, a weight of each synapse in the brain activity state classification model is determined according to the Hebbian information corresponding to each synapse and a backward propagation result. The Hebbian information is determined based on a co-firing frequency of each synapse and is used for representing a degree of association between a training task and each synapse; and the brain activity state classification model is established based on a spiking neural network.

Further, the step of updating the Hebbian information corresponding to each synapse in the brain activity state classification model according to the pulse sequences corresponding to the multiple training tasks includes the step described below.

The Hebbian information corresponding to each synapse in the brain activity state classification model is updated using the formulas described below.

$$\{ \begin{matrix}{H}_{i , j}^{n} = ω {f}_{i , j} + ( 1 - ω ) {H}_{i , j}^{o} \\ {q}_{i , j} = {H}_{i , j}^{n} ( {q}_{i , j} ∈ {Q}_{i} )\end{matrix} .$$

H_{i,j}^{ω} denotes Hebbian information of an i^{th} synapse before a j^{th} task in a pulse sequence, H_{i,j}ⁿ denotes the Hebbian information of the i^{th} synapse after the j^{th} task in the pulse sequence, ω denotes a preset update rate, f_{i,j} denotes a co-firing frequency of the i^{th} synapse in the brain activity state classification model corresponding to the j^{th} task in the pulse sequence, Qᵢ denotes a target list, the Hebbian information of each synapse corresponding to the multiple training tasks is stored in the target list, and q_{i,j} denotes Hebbian information of the i^{th} synapse corresponding to the j^{th} task stored in the target list.

Further, the step of updating the Hebbian information corresponding to each synapse in the brain activity state classification model includes at least one of the following steps described below.

The Hebbian information of each synapse is updated based on a co-firing state of each synapse in a single time window.

The Hebbian information of each synapse is updated based on an average firing rate over multiple time windows.

Further, the step in which in the backward propagation stage in the target rule, the weight of each synapse in the brain activity state classification model is determined according to the Hebbian information corresponding to each synapse and the backward propagation result includes the step described below.

In the backward propagation stage, for any synapse in the brain activity state classification model, in a case where Hebbian information of the synapse is greater than a first threshold, it is determined that the synapse is associated with a task and the weight of the synapse in the brain activity state classification model is locked; and in a case where the Hebbian information of the synapse is less than or equal to the first threshold, the weight of the synapse is modified according to the backward propagation result.

An embodiment of the present disclosure further provides a brain activity state classification method. The method includes the steps described below.

A pulse sequence corresponding to a target electroencephalography signal is acquired.

The pulse sequence corresponding to the target electroencephalography signal is inputted into a brain activity state classification model to obtain a brain activity state classification result. The brain activity state classification model is trained based on the preceding training method of a brain activity state classification model.

An embodiment of the present disclosure further provides a training apparatus of a brain activity state classification model. The apparatus includes an acquisition module and a training module.

The acquisition module is configured to acquire pulse sequences of electroencephalography signal samples corresponding to multiple training tasks for brain activity state classification.

The training module is configured to input the pulse sequences of the electroencephalography signal samples corresponding to the multiple training tasks into an initial brain activity state classification model and train the brain activity state classification model based on a target rule. In a forward propagation stage in the target rule, Hebbian information corresponding to each synapse in the brain activity state classification model is updated according to the pulse sequences corresponding to the multiple training tasks; and in a backward propagation stage in the target rule, a weight of each synapse in the brain activity state classification model is determined according to the Hebbian information corresponding to each synapse and a backward propagation result. The Hebbian information is determined based on a co-firing frequency of each synapse and is used for representing a degree of association between the multiple training tasks and each synapse; and the brain activity state classification model is established based on a spiking neural network.

An embodiment of the present disclosure further provides an electronic device. The electronic device includes a memory, a processor, and a computer program stored in the memory and executable by the processor, when executing the computer program, the processor performs the preceding training method of a brain activity state classification model or the preceding brain activity state classification method.

An embodiment of the present disclosure further provides a non-transitory computer-readable storage medium storing a computer program, where when executed by a processor, the computer program causes the processor to perform the preceding training method of a brain activity state classification model or the preceding brain activity state classification method.

Through the training method and apparatus of a brain activity state classification model, and the device provided in the embodiments of the present disclosure, in a continuous learning process of the pulse sequences of the electroencephalography signal samples corresponding to multiple training tasks, in the forward propagation stage in the target rule, the Hebbian information records the degree of association between the training tasks and the synapses; and in the backward propagation stage in the target rule, the weights of the synapses are determined through the recorded Hebbian information. In this manner, in the continuous learning process of multiple training tasks, the Hebbian information is recorded to protect the information of trained tasks so that the trained tasks can still be accurately recognized, the catastrophic forgetting issue is solved, the trained brain activity state classification model can accurately classify brain activity states, and the efficiency and accuracy of brain activity state classification are improved.

### BRIEF DESCRIPTION OF DRAWINGS

To illustrate the technical schemes in the present disclosure or the technical schemes in the related art more clearly, drawings used in the description of the embodiments or the related art are briefly described below. Apparently, the drawings described below illustrate part of the embodiments of the present disclosure, and those of ordinary skill in the art may obtain other drawings based on the drawings described below on the premise that no creative work is done.

FIG. 1 is a first flowchart of a training method of a brain activity state classification model according to an embodiment of the present disclosure;

FIG. 2 is a second flowchart of a training method of a brain activity state classification model according to an embodiment of the present disclosure;

FIG. 3 is a third flowchart of a training method of a brain activity state classification model according to an embodiment of the present disclosure;

FIG. 4 is a fourth flowchart of a training method of a brain activity state classification model according to an embodiment of the present disclosure;

FIG. 5 is a structural diagram of a training apparatus of a brain activity state classification model according to an embodiment of the present disclosure; and

FIG. 6 is a structural diagram of an electronic device according to an embodiment of the present disclosure.

### DETAILED DESCRIPTION

To illustrate the object, technical schemes, and advantages of the present disclosure more clearly, the technical schemes in the present disclosure are described below clearly and completely in conjunction with the drawings in the present disclosure. Apparently, the embodiments described below are part, not all, of the embodiments of the present disclosure. Based on the embodiments of the present disclosure, all other embodiments acquired by those of ordinary skill in the art on the premise that no creative work is done are within the scope of the present disclosure.

The method in the embodiments of the present disclosure may be applied in medical signal processing scenarios to achieve accurate classification of brain activity states.

In the related art, the artificial neural network is used for classifying brain activity states. However, in a scenario where data distribution continues to change, the artificial neural network model is plagued by the same catastrophic forgetting issue as the traditional methods, that is, the learning of new knowledge interferes with the memory of old knowledge, causing the brain activity state classification result to be less accurate and less efficient.

Through the training method of a brain activity state classification model in the embodiments of the present disclosure, in the continuous learning process of the pulse sequences of the electroencephalography signal samples corresponding to multiple training tasks, in the forward propagation stage in the target rule, the Hebbian information records the degree of association between the training tasks and the synapses; and in the backward propagation stage in the target rule, the weights of the synapses are determined through the recorded Hebbian information. In this manner, in the continuous learning process of multiple training tasks, the Hebbian information is recorded to protect the information of trained tasks so that the trained tasks can still be accurately recognized to solve the catastrophic forgetting issue, the trained brain activity state classification model can accurately classify the brain activity states, and the efficiency and accuracy of brain activity state classification are improved.

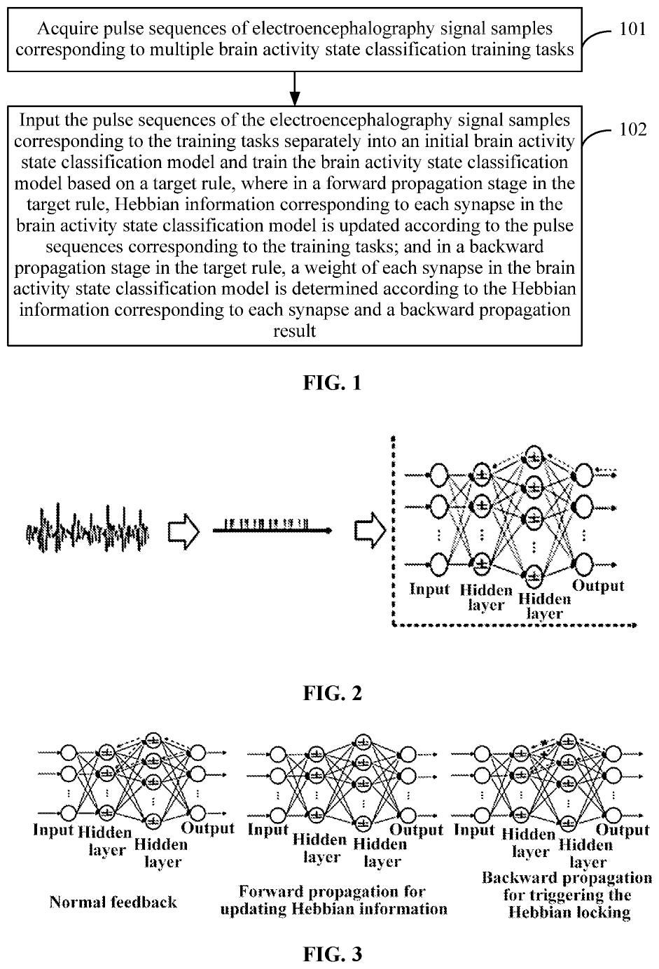

The technical schemes in the present disclosure are described below in detail through the embodiments in conjunction with FIGS. 1 to 6. The embodiments described below may be combined with each other, and identical or similar concepts or processes may not be repeated in some embodiments.

FIG. 1 is a first flowchart of an embodiment of a training method of a brain activity state classification model according to an embodiment of the present disclosure. As shown in FIG. 1, the method in this embodiment includes the steps described below.

In step 101, pulse sequences of electroencephalography signal samples corresponding to multiple training tasks for brain activity state classification are acquired.

In the related art, the artificial neural network is used for classifying brain activity states. However, in a scenario where data distribution continues to change, the artificial neural network model is plagued by the same catastrophic forgetting issue as the traditional methods, that is, the learning of new knowledge interferes with the memory of old knowledge, causing the brain activity state classification result to be less accurate and less efficient.

In the embodiments of the present disclosure, the pulse sequences of the electroencephalography signal samples corresponding to multiple training tasks for brain activity state classification are first acquired. In an embodiment, in the training tasks for brain activity state classification, a heart rate signal, a brain signal, an audio signal, and other signals are inputted, and a non-pulse input signal is encoded into a new pulse sequence by using a pulse encoder (such as a Poisson encoder) for the training of the brain activity state classification model. For example, a segment of heart rate signal input is divided into N frames, and each frame is encoded into a pulse sequence with a normal distribution or another distribution.

In step 102, the pulse sequences of the electroencephalography signal samples corresponding to the multiple training tasks are inputted into an initial brain activity state classification model, and the brain activity state classification model is trained based on a target rule. In a forward propagation stage in the target rule, Hebbian information corresponding to each synapse in the brain activity state classification model is updated according to the pulse sequences corresponding to the multiple training tasks; and in a backward propagation stage in the target rule, a weight of each synapse in the brain activity state classification model is determined according to the Hebbian information corresponding to each synapse and a backward propagation result. The Hebbian information is determined based on the co-firing frequency of each synapse and is used for representing a degree of association between the multiple training tasks and each synapse; and the brain activity state classification model is established based on a spiking neural network.

After the pulse sequences of the electroencephalography signal samples corresponding to multiple training tasks for brain activity state classification are acquired, in the embodiments of the present disclosure, the pulse sequences of the electroencephalography signal samples corresponding to the training tasks for brain activity state classification are inputted into the initial brain activity state classification model for continuous learning, and the brain activity state classification model is trained based on the target rule. In an embodiment, the target rule includes the forward propagation stage and the backward propagation stage, backward propagation is performed according to an error between an actual output value and an expected output value in the forward propagation stage, and the loop iteration is performed for learning and training of brain activity state classification model parameters. After the training is completed, the brain activity state classification model can be used for classifying brain activity states. In the forward propagation stage in the target rule, according to the embodiments of the present disclosure, the Hebbian information corresponding to each synapse in the brain activity state classification model is updated according to the pulse sequences corresponding to the training tasks; and in the backward propagation stage in the target rule, the weight of each synapse in the brain activity state classification model is determined according to the Hebbian information corresponding to each synapse and the backward propagation result. That is, in the continuous learning process of the pulse sequences of the electroencephalography signal samples corresponding to multiple training tasks, in the forward propagation stage in the target rule, the degree of association between the training tasks and the synapses is recorded through the Hebbian information; and in the backward propagation stage in the target rule, the weights of the synapses are determined according to the recorded Hebbian information and the backward propagation result. Thus, in the continuous learning process of multiple training tasks, the Hebbian information is recorded to protect the information of trained tasks so that the trained tasks can still be normally recognized. That is, during the multi-task training, the Hebbian information of the synapses is recorded so that highly active neurons corresponding to different tasks are found, the neurons are respectively allocated as subsystems of the tasks, and the weights of the neurons are locked and remain unchanged in the subsequent learning of other tasks. In this manner, the new training task does not affect the previously trained tasks so that the previous tasks are not forgotten, and a model and method that can achieve efficient training and adaptively allocate neurons to form subsystems without clear multi-task information are innovatively provided, thereby solving the catastrophic forgetting issue.

For example, the pulse sequence of the electroencephalography signal sample corresponding to the first training task is the corresponding pulse sequence when a user watches a picture, and the first training task corresponds to the first type of brain activity. In the forward propagation stage in the target rule, according to the first training task, Hebbian information of synapse A in the brain activity state classification model is recorded as a; and in the backward propagation stage in the target rule, a change amount of the weight of synapse A is determined according to the backward propagation result and Hebbian information a corresponding to synapse A. The pulse sequence of the electroencephalography signal sample corresponding to the second training task is the corresponding pulse sequence when the user listens to audio, and the second training task corresponds to the second type of brain activity. In the forward propagation stage in the target rule, according to the second training task, Hebbian information of synapse B in the brain activity state classification model is recorded as b; and in the backward propagation stage in the target rule, a weight of synapse B in the brain activity state classification model is determined according to the backward propagation result and Hebbian information b corresponding to synapse B. That is, in the forward propagation stage in the target rule, the Hebbian information records the degree of association between the training tasks and the synapse; and in the backward propagation stage in the target rule, the change amount of the weight of the synapse is determined according to the backward propagation result and the Hebbian information. After the second training task is completed, the brain activity state classification model can still accurately classify the first type of brain activity. That is, in the continuous learning process of multiple training tasks, the Hebbian information is recorded to protect the information of trained tasks so that the trained tasks can still be accurately recognized. That is, in the case of multiple tasks, the new training task does not affect the previously trained tasks so that the previous tasks are not forgotten, thereby solving the catastrophic forgetting issue.

It is to be noted that in the related art, the neural network is modularized, and subsystems containing quantitative neurons are randomly allocated to different tasks. From a biological perspective, this is more in line with the brain's characteristics for multi-task continuous learning (for example, memory and motion control are controlled by different brain areas). However, there are some problems with this paradigm. The first one is the subsystem training efficiency. Since the neural network randomly allocates the subsystem formed by quantitative neurons to each task for training, it means that in a case where the number of tasks is too large or the task training volume is too large, for example, the high-throughput multi-modal dataflow such as electroencephalography signals is inputted, the training data is unbalanced relative to the number of neurons, the subsystem training efficiency is too low, so that the entire network training efficiency is too low. The second one with the training efficiency is that this modular architecture requires knowing the number of tasks and the training sequence in advance to divide the subsystems for the tasks. This means that without knowing the number of tasks and the learning sequence in advance, it is difficult to use this modular architecture paradigm for multi-task training, such as an electroencephalography signal classification task in which the sequence and number cannot be determined. In the embodiment of the present disclosure, the continuous learning of the pulse sequences of the electroencephalography signal samples corresponding to multiple training tasks for brain activity state classification is performed; in the forward propagation stage in the target rule, the Hebbian information records the degree of association between the training tasks and the synapses; and in the backward propagation stage in the target rule, the weights of the synapses are determined through the recorded Hebbian information and the backward propagation result. Thus, in the continuous learning process of multiple training tasks, the Hebbian information is recorded to protect the information of trained tasks so that the trained tasks can still be accurately recognized, and the previous tasks are not forgotten, thereby solving the catastrophic forgetting issue. Compared with the modular training process of the neural network, the embodiments of the present disclosure provide a stronger continuous learning capability. When allocating the subsystems to continuous learning tasks, the present disclosure adopts adaptive calculation and allocation. Compared with the deep neural network and the traditional modular architecture continuous learning paradigm, the present disclosure has a stronger continuous learning capability and can complete the multi-task training more efficiently. In the embodiments of the present disclosure, the entire network is used for the learning of different tasks, and multi-task and large-task training are more efficient, which is a capability that the traditional modular architecture continuous learning paradigm cannot possess.

In addition, the spiking neural network has more complex neurons and synaptic structures than the deep neural network, and many biological rules ignored by the existing artificial networks are exactly the key to achieving general human-like brain intelligence. Therefore, these biological rules are added to the more brain-like spiking neural network so that the current network obtains more powerful computing power and adaptability. In the embodiments of the present application, the brain activity state classification model is established based on the spiking neural network so that the brain activity state classification model design and the continuous learning method are more biologically reasonable. In the embodiment of the present disclosure, during the multi-task training, the Hebbian information of the synapses is recorded so that highly active neurons corresponding to different tasks are found, these neurons are respectively allocated as subsystems of the tasks, and the weights of the neurons are locked and remain unchanged in the subsequent learning of other tasks. In this manner, a model and method that can achieve efficient training and adaptively allocate neurons to form subsystems without clear multi-task information is innovatively provided, thereby solving the two problems existing in the modular architecture paradigm and greatly enhancing the continuous learning capability of the spiking neural network.

Through the method in the preceding embodiment, in the continuous learning process of the pulse sequences of the electroencephalography signal samples corresponding to multiple training tasks, the Hebbian information records the degree of association between the training tasks and the synapses in the forward propagation stage in the target rule; and the weights of the synapses are determined through the recorded Hebbian information and the backward propagation result in the backward propagation stage in the target rule. Thus, in the continuous learning process of multiple training tasks, the Hebbian information is recorded to protect the information of trained tasks so that the trained tasks can still be accurately recognized, the catastrophic forgetting issue is solved, the trained brain activity state classification model can accurately classify the brain activity states, and the efficiency and accuracy of brain activity state classification are improved.

In an embodiment, the step of updating the Hebbian information corresponding to each synapse in the brain activity state classification model according to the pulse sequences corresponding to the multiple training tasks includes the following.

The Hebbian information corresponding to each synapse in the brain activity state classification model is updated using the formulas described below.

$$\{ \begin{matrix}{H}_{i , j}^{n} = ω {f}_{i , j} + ( 1 - ω ) {H}_{i , j}^{o} \\ {q}_{i , j} = {H}_{i , j}^{n} ( {q}_{i , j} ∈ {Q}_{i} )\end{matrix} .$$

H_{i,j}^{ω} denotes Hebbian information of an i^{th} synapse before a j^{th} task in a pulse sequence, H_{i,j}ⁿ denotes Hebbian information of the i^{th} synapse after the j^{th} task in the pulse sequence, ω denotes a preset update rate, f_{i,j} denotes a co-firing frequency of the i^{th} synapse in the brain activity state classification model corresponding to the j^{th} task in the pulse sequence, Qᵢ denotes a target list, the Hebbian information of each synapse corresponding to the multiple training tasks is stored in the target list, and q_{i,j} denotes the Hebbian information of the i^{th} synapse corresponding to the j^{th} task stored in the target list.

In the embodiments of the present disclosure, the continuous learning of the pulse sequences of the electroencephalography signal samples corresponding to multiple training tasks for brain activity state classification is performed; the Hebbian information records the degree of association between the training tasks and the synapses in the forward propagation stage in the target rule; and the weights of the synapses are determined through the recorded Hebbian information and the backward propagation result in the backward propagation stage in the target rule. Thus, in the continuous learning process of multiple tasks, the Hebbian information is recorded to protect the information of trained tasks so that the trained tasks can still be normally recognized, and the previous tasks are not forgotten, thereby solving the catastrophic forgetting issue. In an embodiment, the Hebbian information corresponding to each synapse in the brain activity state classification model is updated and recorded using the formulas described below.

$$\{ \begin{matrix}{H}_{i , j}^{n} = ω {f}_{i , j} + ( 1 - ω ) {H}_{i , j}^{o} \\ {q}_{i , j} = {H}_{i , j}^{n} ( {q}_{i , j} ∈ {Q}_{i} )\end{matrix} .$$

H_{i,j}^{ω} denotes Hebbian information of an i^{th} synapse before a j^{th} task in a pulse sequence, H_{i,j}ⁿ denotes Hebbian information of the i^{th} synapse after the j^{th} task in the pulse sequence, ω denotes a preset update rate, f_{i,j} denotes the co-firing frequency of the i^{th} synapse in the brain activity state classification model corresponding to the j^{th} task in the pulse sequence, Qᵢ denotes a target list, the Hebbian information of each synapse corresponding to the multiple training tasks is stored in the target list, and q_{i,j} denotes the Hebbian information of the i^{th} synapse corresponding to the j^{th} task stored in the target list. That is, a variable is defined for the synapse to describe the frequency of the co-firing phenomenon, and this variable is referred to as the Hebbian information. In the forward propagation stage of the training of each task, each synapse calculates, updates, and records the Hebbian information corresponding to the task. The processing method is as follows: all tasks are inputted into the network in sequence for learning in a continuous learning paradigm; during the learning process of each task, only data of the task is presented, and data of historical tasks is not presented. In the forward propagation stage of each task, each synapse calculates and updates the Hebbian information of the corresponding task using the formulas described below.

$$\{ \begin{matrix}{H}_{i , j}^{n} = ω {f}_{i , j} + ( 1 - ω ) {H}_{i , j}^{o} \\ {q}_{i , j} = {H}_{i , j}^{n} ( {q}_{i , j} ∈ {Q}_{i} )\end{matrix} .$$

ω denotes the update rate, H_{i,j}^{ω} and H_{i,j}ⁿ separately denote the Hebbian information of the i^{th} synapse before and after the update in the forward propagation stage of the j^{th} task, and f_{i,j} denotes the co-firing frequency of each synapse in the forward propagation stage of the current task. ω is an artificially set parameter, and the initialization value of H_{i,j}^{o} is 0. Qᵢ denotes a list of the i^{th} synapse for storing Hebbian information corresponding to the historical tasks, and q_{i,j} denotes the Hebbian information corresponding to the j^{th} task stored in the list. That is, during the multi-task training, the Hebbian information of the synapse is recorded for each task so that highly active neurons corresponding to different tasks are found, these neurons are respectively allocated as subsystems of the tasks, and the weights of these neurons are locked and remain unchanged in the subsequent learning of other tasks. Thus in the continuous learning process of multiple training tasks, the Hebbian information is recorded to protect the information of trained tasks so that the trained tasks can still be normally recognized, the catastrophic forgetting issue is solved, the trained brain activity state classification model can accurately classify the brain activity states, and the efficiency and accuracy of brain activity state classification are improved.

In the method of the preceding embodiment, all tasks are inputted into the brain activity state classification model in sequence for learning in a continuous learning paradigm. In the forward propagation stage of the training of each task, each synapse calculates, updates, and records the Hebbian information corresponding to the task. That is, during the multi-task training, the Hebbian information of the synapse is recorded for each task so that highly active neurons corresponding to different tasks are found, the neurons are respectively allocated as subsystems of the tasks, and the weights of the neurons are locked and remain unchanged in the subsequent learning of other tasks. Thus, in the continuous learning process of multiple training tasks, the Hebbian information is recorded to protect the information of trained tasks so that the trained tasks can still be normally recognized, the catastrophic forgetting issue is solved, the trained brain activity state classification model can accurately classify the brain activity states, and the efficiency and accuracy of brain activity state classification are improved.

In an embodiment, the step of updating the Hebbian information corresponding to each synapse in the brain activity state classification model includes at least one of the following:
The Hebbian information of each synapse is updated based on the co-firing state of each synapse in a single time window; and/or
the Hebbian information of each synapse is updated based on an average firing rate over multiple time windows.

The Hebbian information of each synapse may be updated based on two methods. The first method is to update the Hebbian information according to neuron activity information in several time windows, that is, in the forward propagation stage, the Hebbian information is updated according to the average firing rate over multiple time windows. f_{i,j} is expressed as follows:

$${f}_{i , j} = \frac{{\sum }_{t = 1}^{T} {S}_{t}^{p r e}}{T} \cdot \frac{{\sum }_{t = 1}^{T} {S}_{t}^{post}}{T} .$$

S^{pre}ₜ and S^{post}ₜ separately denote the firing states of a presynaptic neuron and a postsynaptic neuron in a t^{th} time window. In this case, the Hebbian information is updated every T time windows.

The second method is to update and calculate the Hebbian information according to the co-firing state of the synapse in a single time window, that is, in the forward propagation stage, the Hebbian information is updated according to the neuron activity information in a single time window. f_{i,j} is expressed as follows:

$${f}_{i , j} = {S}_{t}^{pre} \cdot {S}_{t}^{post} .$$

In this case, the Hebbian information is updated every time window.

The more active the activity of the neuron before and after the i^{th} synapse, the more frequent the co-firing phenomenon, the larger f_{i,j}, and the larger the updated Hebbian information, which means that the i^{th} synapse is more important for the j^{th} task.

In the method of the preceding embodiment, the Hebbian information is updated through the neuron activity information in multiple time windows, or the Hebbian information is updated according to the co-firing state of each synapse in a single time window. In this manner, the Hebbian information is updated timely and accurately so that the more active the synaptic activity corresponding to the training task is, the larger the updated Hebbian information is, and the more important the synapse is for the training task. Therefore, highly active synapses corresponding to different tasks are found, the synapses are respectively allocated as subsystems of the tasks, and the weights of the synapses are locked and remain unchanged in the subsequent learning of other tasks. Thus, in the continuous learning process of multiple training tasks, the Hebbian information is recorded to protect the information of trained tasks so that the trained tasks can still be normally recognized, the catastrophic forgetting issue is solved, the trained brain activity state classification model can accurately classify the brain activity states, and the efficiency and accuracy of brain activity state classification are improved.

In an embodiment, the step in which the weight of each synapse in the brain activity state classification model is determined according to the Hebbian information corresponding to each synapse and the backward propagation result in the backward propagation stage in the target rule includes the step described below.

In the backward propagation stage, for any synapse in the brain activity state classification model, in a case where Hebbian information of the synapse is greater than a first threshold, it is determined that the synapse is associated with the task and the weight of the synapse in the brain activity state classification model is locked; and in a case where the Hebbian information of the synapse is less than or equal to the first threshold, the weight of the synapse is modified according to the backward propagation result.

In the embodiments of the present disclosure, the continuous learning of the pulse sequences of the electroencephalography signal samples corresponding to multiple training tasks for brain activity state classification is performed; the Hebbian information records the degree of association between the training tasks and the synapses in the forward propagation stage in the target rule; and the weights of the synapses are determined through the recorded Hebbian information and the backward propagation result in the backward propagation stage in the target rule. Thus, in the continuous learning process of multiple training tasks, the Hebbian information is recorded to protect the information of trained tasks so that the trained tasks can still be normally recognized, and the previous tasks are not forgotten, thereby solving the catastrophic forgetting issue. In an embodiment, a Hebbian synaptic lock operation is performed according to the Hebbian information in the backward propagation stage of the neural network. In the backward propagation stage, a mask for masking is generated for the synapses according to Hebbian information accumulated from the recorded historical tasks, thereby protecting the knowledge related to the historical tasks in the network and improving the continuous learning capability of the network. In the backward propagation stage of each task, it is determined whether the synapse is associated with a certain historical task according to the Hebbian information corresponding to the historical tasks recorded by each synapse in the forward propagation stage. The association criterion of the i^{th} synapse is calculated as follows:

$$\{ \begin{matrix}{q}_{i , j}^{m} = max ( {Q}_{i} ) \\ {P}_{i} = 0 , if ( {q}_{i , j}^{m} > {q}_{t h} )\end{matrix} .$$

q_{i,j}^{m} denotes the maximum Hebbian information value corresponding to the j^{th} task in the list of the i^{th} synapse for storing the Hebbian information corresponding to the historical tasks, and Pᵢ denotes an association mark. If the maximum Hebbian information value q_{i,j}^{m} is greater than a threshold qₜₕ, the i^{th} synapse is considered to be associated with the j^{th} task. During the backward propagation, the change amount of the i^{th} synapse is masked through a mask to ensure that the weight of the associated synapse i is not changed by the current task, that is, the weight of the synapse is locked; otherwise, the backward propagation is performed according to the error between the actual output value and the expected output value in the forward propagation stage, and the loop iteration is performed for learning and training of brain activity state classification model parameters. The association determination between the synapses and the tasks and the synapse masking method are the main content of the Hebbian synaptic lock, thereby achieving continuous learning of multiple tasks. In the forward propagation stage of the training of each task, each synapse calculates, updates, and records the Hebbian information corresponding to the task so that highly active neurons corresponding to different tasks are found, these neurons are respectively allocated as subsystems of the tasks, and the weights of the neurons are locked and remain unchanged in the subsequent learning of other tasks. Thus, in the continuous learning process of multiple training tasks, the Hebbian information is recorded to protect the information of trained tasks so that the trained tasks can still be normally recognized, the catastrophic forgetting issue is solved, the trained brain activity state classification model can accurately classify the brain activity states, and the efficiency and accuracy of brain activity state classification are improved.

In the method of the preceding embodiments, each synapse calculates, updates, and records the Hebbian information corresponding to the tasks in the forward propagation stage of the training of each task. That is, during the multi-task training, the Hebbian information of the synapse is recorded for each task so that highly active neurons corresponding to different tasks are found; in the backward propagation stage, the highly active neurons are respectively allocated as subsystems of the tasks, and the weights of the neurons are locked and remain unchanged in the subsequent learning of other tasks. Thus, in the continuous learning process of multiple training tasks, the Hebbian information is recorded to protect the information of trained tasks so that the trained tasks can still be normally recognized, the catastrophic forgetting issue is solved, the trained brain activity state classification model can accurately classify the brain activity states, and the efficiency and accuracy of brain activity state classification are improved.

In an embodiment, a brain activity state classification method includes the steps described below.

A pulse sequence corresponding to a target electroencephalography signal is acquired.

The pulse sequence corresponding to the target electroencephalography signal is inputted into a brain activity state classification model to obtain a brain activity state classification result. The brain activity state classification model is trained based on the training method of the brain activity state classification model.

In the embodiments of the present disclosure, the Hebbian information is recorded to protect the information of trained tasks in the continuous learning process of multiple training tasks so that the trained tasks can still be normally recognized, the catastrophic forgetting issue is solved, the trained brain activity state classification model can accurately classify the brain activity states, and the efficiency and accuracy of brain activity state classification are improved. In an embodiment, after the brain activity state classification model is trained, the pulse sequence corresponding to a to-be-recognized electroencephalography signal may be inputted into the brain activity state classification model to obtain the brain activity state classification result so that accurate brain activity state recognition and classification are achieved, thereby based on accurately recognized and classified brain activity states, the doctor can be assisted in determining the source of a signal and confirming the brain state and physical condition of the patient for more accurate treatment.

In the method of the preceding embodiments, the pulse sequence corresponding to the to-be-recognized electroencephalography signal is inputted into the trained brain activity state classification model so that the brain activity state classification result is accurately obtained, and the brain activity state can be accurately recognized.

For example, in the flowchart of the training method of the brain activity state classification model shown in FIG. 2, the continuous learning model and method of the spiking neural network based on the Hebbian synaptic lock can achieve a stronger continuous learning capability and higher training efficiency, and at the same time, a more biocredible neural network learning model and method are provided below.
(1) Input data is encoded into a pulse sequence. A heart rate signal, a brain signal, an audio signal, and other signals are inputted, a non-pulse input signal is encoded into a new pulse sequence in a certain distribution form by using a pulse encoder (such as a Poisson encoder), and the new pulse sequence is used and processed by subsequent spiking neurons. For example, a segment of heart rate signal input is divided into N frames, and each frame is encoded into a pulse sequence with a normal distribution or another distribution.
(2) Dynamic neurons with predefined thresholds process pulse information. The dynamic neurons encode input information and determine dynamic characteristics according to a predefined neuron firing threshold. The process in which basic Leaky Integrate-and-Fire (LIF) neurons process information at the current moment is described as follows:

$$\begin{matrix}C \frac{{dV}_{i} ( t )}{dt} = g ( {V}_{i} ( t ) - {V}_{rest} ) ( 1 - S ) + {\sum }_{j = 1}^{N} {W}_{i , j} {X}_{j} ( t ) \\ {V}_{i} ( t ) = {V}_{rest} , S = 1 , if ( {V}_{i} ( t ) = {V}_{t h} ) \\ S = 1 , if ( t - {t}_{s p i k e} < {τ}_{ref} ) , t ∈ ( 1 , {T}_{1} )\end{matrix} .$$

Vᵢ(t) denotes a membrane potential with a historical integration state, S denotes a neuron firing state, and S=1 denotes the pulse when the membrane potential Vᵢ(t) of a neuron i reaches the firing threshold Vₜₕ. At the same time, S is used for simulating the refractory period τ_{ref} of the neuron by resetting the membrane potential rather than directly blocking the membrane potential.

Based on the LIF neurons, the neuron firing threshold is an artificially set static value and is determined by the required neuron dynamic characteristics.
(3) The dynamic neurons are used to establish a spiking neural network with adaptive Hebbian information calculation. A variable is defined for the synapse to describe the frequency of the co-firing phenomenon, and this variable is referred to as the Hebbian information. In the forward propagation stage of the training of each task, each synapse calculates, updates, and records the Hebbian information corresponding to the task. The processing method is described below.

In the flowchart of the training method of the brain activity state classification model shown in FIG. 3, all tasks are inputted into the network in sequence for learning in a continuous learning paradigm; during the learning process of each task, only the data of the current task is presented, and the data of historical tasks is not presented. In the forward propagation stage of each task, each synapse calculates and updates the Hebbian information of the corresponding task using the following formulas:

$$\{ \begin{matrix}{H}_{i , j}^{n} = ω {f}_{i , j} + ( 1 - ω ) {H}_{i , j}^{o} \\ {q}_{i , j} = {H}_{i , j}^{n} ( {q}_{i , j} ∈ {Q}_{i} )\end{matrix} .$$

ω denotes the update rate, H_{i,j}^{o} and H_{i,j}ⁿ separately denote the Hebbian information of the i^{th} synapse before and after the update in the forward propagation stage of the j^{th} task, and f_{i,j} denotes the co-firing frequency of each synapse in the forward propagation stage of the current task and is calculated through two technical routes for updating the Hebbian information. ω is an artificially set parameter, and the initialization value of H_{i,j}^{o} is 0. Qᵢ denotes a list of the i^{th} synapse for storing the Hebbian information corresponding to the historical tasks, and q_{i,j} denotes the Hebbian information corresponding to the j^{th} task stored in the list.

The Hebbian information of each synapse may be updated based on two methods. The first method is to update the Hebbian information according to the neuron activity information in multiple time windows. That is, f_{i,j} is expressed below in this case.

$${f}_{i , j} = \frac{{\sum }_{t = 1}^{T} {S}_{t}^{p r e}}{T} \cdot \frac{{\sum }_{t = 1}^{T} {S}_{t}^{post}}{T} .$$

S^{pre}ₜ and S^{post}ₜ separately denote the firing states of presynaptic and postsynaptic neurons in the t^{th} time window. In this case, the Hebbian information is updated every T time windows.

The second method is to update and calculate the Hebbian information according to the co-firing state of the synapse in a single time window. That is, f_{i,j} is expressed below in this case.

$${f}_{i , j} = {S}_{t}^{pre} \cdot {S}_{t}^{post} .$$

In this case, the Hebbian information is updated every time window.

In an embodiment, the more active the activity of the neuron before and after the i^{th} synapse, the more frequent the co-firing phenomenon, the larger f_{i,j}, and the larger the updated Hebbian information, which means that the i^{th} synapse is more important for the j^{th} task.
(4) The Hebbian synaptic lock operation is performed according to the Hebbian information in the backward propagation stage. In the backward propagation stage, a mask for masking is generated for the synapses according to the Hebbian information accumulated from the recorded historical tasks, thereby protecting the knowledge related to the historical tasks in the network and improving the continuous learning capability. In the backward propagation stage of each task, it is determined whether a synapse is associated with a certain historical task according to the Hebbian information corresponding to the historical tasks recorded by each synapse. The association criterion of the i^{th} synapse is calculated through Q; in the formula described below.

$$\{ \begin{matrix}{q}_{i , j}^{m} = max ( {Q}_{i} ) \\ {P}_{i} = 0 , if ( {q}_{i , j}^{m} > {q}_{t h} )\end{matrix} .$$

q_{i,j}^{m} denotes the maximum Hebbian information value corresponding to the j^{th} task in the list of the i^{th} synapse for storing the Hebbian information corresponding to the historical tasks, and Pᵢ denotes the association mark. If the maximum Hebbian information value q_{i,j}^{m} is greater than the threshold qₜₕ, the i^{th} synapse is considered to be associated with the j^{th} task. During the backward propagation, the change amount of the i^{th} synapse is masked through a mask to ensure that the weight of the associated synapse i is not changed by the current task, that is, the weight of the synapse is locked. Here, the association determination between the synapses and the tasks and the synapse masking method are considered as the main content of the Hebbian synaptic lock.
(5) The spiking neural network continuous learning model based on the Hebbian synaptic lock is used to recognize sequences such as a heart rate and a brain signal. That is, the trained brain activity state classification model is used to recognize sequence information such as a heart rate and a brain signal in a manner of group decision-making in an output layer. For one input, the category with the most responses is used as the final output category of the model classification.

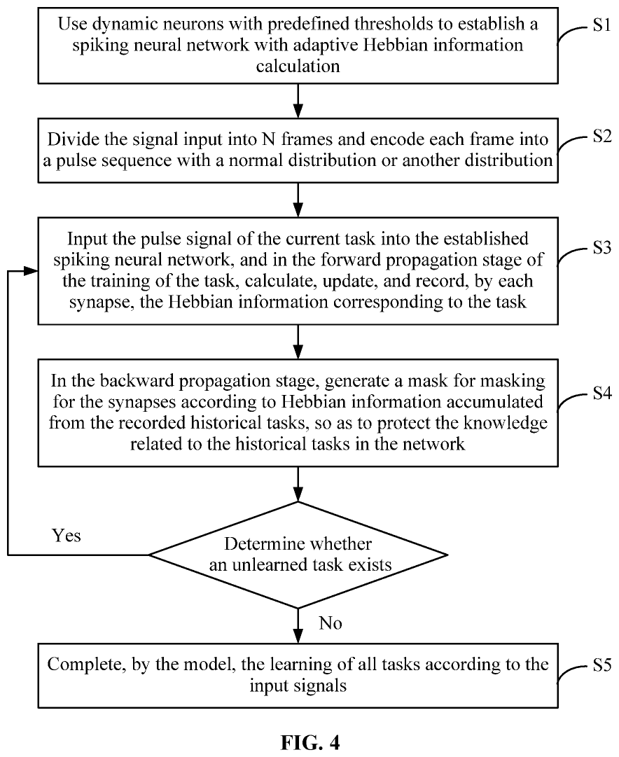

For example, the flowchart of the training method of the brain activity state classification model shown in FIG. 4 is described below.

In step S1, dynamic neurons with predefined thresholds are used to establish the spiking neural network with adaptive Hebbian information calculation, and then the initial brain activity state classification model is established based on the spiking neural network.

In step S2, the inputted signals, that is, the electroencephalography signal samples corresponding to multiple training tasks for the brain activity state classification, are divided into N frames, and each frame is encoded into a pulse sequence with a normal distribution or another distribution.

In step S3, the pulse signal of the current task is inputted into the established initial brain activity state classification model, and in the forward propagation stage of the training of the task, each synapse calculates, updates, and records the Hebbian information corresponding to the task.

In step S4, in the backward propagation stage, a mask for masking is generated for the synapses according to the Hebbian information accumulated from the recorded historical tasks, thereby protecting the knowledge related to the historical tasks in the network. The information of trained tasks is protected through the Hebbian information so that the trained tasks can still be accurately recognized, thereby solving the catastrophic forgetting issue.

In step S5, it is determined whether an unlearned task exists; if the unlearned task exists, steps S3 and S4 are repeated until the initial brain activity state classification model completes the learning of all the tasks to train the brain activity state classification model.

For example, the Modified National Institute of Standards and Technology database (MNIST database) is selected for Task-IL continuous learning task verification. Task_IL is task incremental learning. In this scenario, both in a training stage or a testing stage, the model is informed of the current task ID, and different tasks have independent output layers. The preceding classification learning method is adopted to verify the relationships between the average accuracy rate and each of the network size, the firing sparsity, and the synaptic locking ratio. The accuracy rate is defined as the number of accurately recognized samples divided by the total number of samples. The threshold is defined as the proportion of locked synapses. The verification result shows that the method of the present disclosure has a higher accuracy advantage in Task-IL continuous learning, and the variation relationships between the average accuracy rate and the three parameters all satisfy the properties of the network we designed.

For example, the MNIST database is selected for Domain-IL continuous learning task verification. Domain_IL is domain incremental learning. Compared with Task-IL, a new restriction is added in the testing stage, that is, the task ID is not known during a prediction stage, and different tasks share the same output layer. The model needs to accurately classify the data without knowing the task TD. The preceding classification learning method is adopted to verify the relationships between the average accuracy rate and each of the network size, the firing sparsity, and the synaptic locking ratio. The accuracy rate is defined as the number of accurately recognized samples divided by the total number of samples. The verification result shows that the variation relationships between the average accuracy rate and the three parameters are very apparent and in line with the properties of the network established in the present disclosure.

The settings of parameters in the preceding two examples are shown in Table 1.

TABLE 1

| | | | Neuronal | | Time window | |
|---|---|---|---|---|---|---|
| | | | firing | | T for | |
| | Learning | Conductivity | threshold | Refractory | dynamic | |
| Tasks | rate | g | Vₜₕ | period τ_{ref} | neurons | Window |
| Task-IL | 1e−4 | 0.2 nS | 0.5 mV | 1 ms | 10-100 ms | 0.5 mV |
| continuous | | | | | | |
| learning | | | | | | |
| task | | | | | | |
| Domain-IL | 1e−4 | | | | | |
| continuous | | | | | | |
| learning | | | | | | |
| task | | | | | | |

g denotes the conductivity, Vₜₕ denotes the neuronal firing threshold, T_{ref} denotes the refractory period, and T denotes the time window for simulating dynamic neurons. Further, in the present disclosure, the capacitance C of the membrane potential is equal to 1 μF/cm², and the reset membrane potential Vᵣₑₛₜ is equal to 0 mV.

It can be seen that the present disclosure has the advantages described below.

The method in the present disclosure has a stronger continuous learning capability. When allocating the subsystems for continuous learning tasks, the present disclosure adopts adaptive calculation and allocation. Compared with the deep neural network and the traditional modular architecture continuous learning paradigm, the present disclosure has a stronger continuous learning capability.

The present disclosure can complete the multi-task training more efficiently. In the present disclosure, the entire network is used for the learning of different tasks, and multi-task and large-task training are more efficient, which is a capability that the traditional modular architecture continuous learning paradigm cannot possess.

The present disclosure has the biological reasonability. In the present disclosure, the synapse selection based on the Hebbian theory and the Hebbian synaptic lock, and the adaptive allocation of task subsystems make the model design and the continuous learning method more biologically reasonable.

A training apparatus of a brain activity state classification model provided in the present disclosure is described below. The training apparatus of the brain activity state classification model described below and the training method of the brain activity state classification model described above may be mutually referenced.

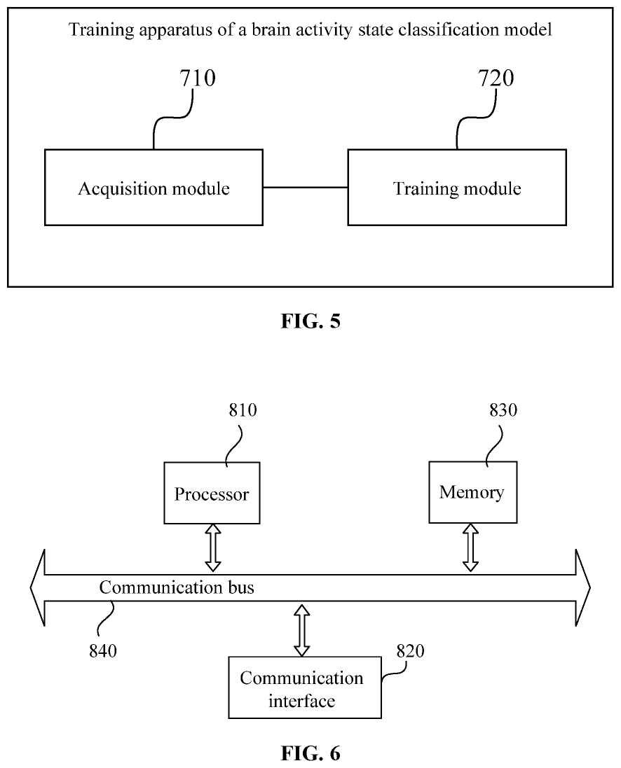

FIG. 5 is a structural diagram of a training apparatus of a brain activity state classification model according to the present disclosure. The training apparatus of the brain activity state classification model provided in this embodiment includes an acquisition module 710 and a training module 720.

The acquisition module 710 is configured to acquire pulse sequences of electroencephalography signal samples corresponding to multiple training tasks for brain activity state classification.

The training module 720 is configured to input the pulse sequences of the electroencephalography signal samples corresponding to the multiple training tasks into an initial brain activity state classification model and train the brain activity state classification model based on a target rule, where in a forward propagation stage in the target rule, Hebbian information corresponding to each synapse in the brain activity state classification model is updated according to the pulse sequences corresponding to the multiple training tasks; and in a backward propagation stage in the target rule, a weight of each synapse in the brain activity state classification model is determined according to the Hebbian information corresponding to each synapse and a backward propagation result; the Hebbian information is determined based on a co-firing frequency of each synapse; the Hebbian information is used for representing a degree of association between the multiple training tasks and each synapse; and the brain activity state classification model is established based on a spiking neural network.

In an embodiment, the training module 720 is configured to update the Hebbian information corresponding to each synapse in the brain activity state classification model using the formulas described below.

$$\{ \begin{matrix}{H}_{i , j}^{n} = ω {f}_{i , j} + ( 1 - ω ) {H}_{i , j}^{o} \\ {q}_{i , j} = {H}_{i , j}^{n} ( {q}_{i , j} ∈ {Q}_{i} )\end{matrix} .$$

H_{i,j}^{o} denotes Hebbian information of an i^{th} synapse before a j^{th} task in the pulse sequence, H_{i,j}ⁿ denotes Hebbian information of the i^{th} synapse after the j^{th} task in the pulse sequence, ω denotes a preset update rate, f_{i,j} denotes the co-firing frequency of the i^{th} synapse in the brain activity state classification model corresponding to the j^{th} task in the pulse sequence, Qᵢ denotes a target list, the Hebbian information of each synapse corresponding to the multiple training tasks is stored in the target list, and q_{i,j} denotes the Hebbian information of the i^{th} synapse corresponding to the j^{th} task stored in the target list.

In an embodiment, the training module 720 is configured to update the Hebbian information of each synapse based on the co-firing state of each synapse in a single time window.

Moreover/alternatively, the training module 720 is configured to update the Hebbian information of each synapse based on an average firing rate over multiple time windows.

In an embodiment, the training module 720 is configured to, in the backward propagation stage, for any synapse in the brain activity state classification model, in a case where the Hebbian information of the synapse is greater than a first threshold, determine that the synapse is associated with a task and lock the weight of the synapse in the brain activity state classification model; and in a case where the Hebbian information of the synapse is less than or equal to the first threshold, modify the weight of the synapse according to the backward propagation result.

The apparatus in the embodiment of the present disclosure is used for performing the method in any one of the preceding method embodiments. The implementation principle and technical effects are similar and thus are not repeated herein.

FIG. 6 is a schematic diagram of the physical structure of an electronic device. The electronic device may include a processor 810, a communication interface 820, a memory 830, and a communication bus 840, where the processor 810, the communication interface 820, and the memory 830 communicate with each other through the communication bus 840. The processor 810 may call logic instructions in the memory 830 to perform the training method of the brain activity state classification model. The method includes acquiring pulse sequences of electroencephalography signal samples corresponding to multiple training tasks for brain activity state classification; and inputting the pulse sequences of the electroencephalography signal samples corresponding to the multiple training tasks into an initial brain activity state classification model and training the brain activity state classification model based on a target rule. In a forward propagation stage in the target rule, Hebbian information corresponding to each synapse in the brain activity state classification model is updated according to the pulse sequences corresponding to the multiple training tasks; and in a backward propagation stage in the target rule, a weight of each synapse in the brain activity state classification model is determined according to the Hebbian information corresponding to each synapse and a backward propagation result. The Hebbian information is determined based on the co-firing frequency of each synapse; the Hebbian information is used for representing a degree of association between the multiple training tasks and each synapse; and the brain activity state classification model is established based on a spiking neural network.

In addition, the logic instructions in the memory 830 may be implemented in the form of a software function unit and, when sold or used as an independent product, may be stored in a computer-readable storage medium. Based on this understanding, the technical schemes provided in the present disclosure substantially, the part contributing to the related art, or part of the technical schemes, may be embodied in the form of a software product. The computer software product is stored in a storage medium and includes several instructions for enabling a computer device (which may be a personal computer, a server, a network device, or the like) to perform all or part of the steps in the methods provided in the embodiments of the present disclosure. The storage medium includes a Universal Serial Bus (USB) flash disk, a mobile hard disk, a read-only memory (ROM), a random-access memory (RAM), a magnetic disk, an optical disc, or another medium capable of storing program codes.

The present disclosure further provides a computer program product. The computer program product includes a computer program stored in a non-transitory computer-readable storage medium. The computer program includes program instructions, where when the program instructions are executed by a computer, the computer performs the training method of the brain activity state classification model provided in the preceding embodiments. The method includes acquiring pulse sequences of electroencephalography signal samples corresponding to multiple training tasks for brain activity state classification; and inputting the pulse sequences of the electroencephalography signal samples corresponding to the multiple training tasks into an initial brain activity state classification model and training the brain activity state classification model based on a target rule. In a forward propagation stage in the target rule, Hebbian information corresponding to each synapse in the brain activity state classification model is updated according to the pulse sequences corresponding to the multiple training tasks; and in a backward propagation stage in the target rule, a weight of each synapse in the brain activity state classification model is determined according to the Hebbian information corresponding to each synapse and a backward propagation result. The Hebbian information is determined based on the co-firing frequency of each synapse and used for representing the degree of association between the multiple training tasks and each synapse; and the brain activity state classification model is established based on a spiking neural network.

The present disclosure further provides a non-transitory computer-readable storage medium storing a computer program, where when executed by a processor, the computer program causes the processor to perform the training method of the brain activity state classification model described above. The method includes acquiring pulse sequences of electroencephalography signal samples corresponding to multiple training tasks for brain activity state classification; and inputting the pulse sequences of the electroencephalography signal samples corresponding to the multiple training tasks into an initial brain activity state classification model and training the brain activity state classification model based on a target rule. In a forward propagation stage in the target rule, Hebbian information corresponding to each synapse in the brain activity state classification model is updated according to the pulse sequences corresponding to the multiple training tasks; and in a backward propagation stage in the target rule, a weight of each synapse in the brain activity state classification model is determined according to the Hebbian information corresponding to each synapse and a backward propagation result. The Hebbian information is determined based on the co-firing frequency of each synapse and used for representing the degree of association between the multiple training tasks and each synapse; and the brain activity state classification model is established based on a spiking neural network.

The apparatus embodiment is described illustratively. Units described as separate components in the apparatus embodiment may or may not be physically separated. Components presented as units in the apparatus embodiment may or may not be physical units, that is, may be located in one place or may be distributed over multiple network units. Part or all of these modules may be selected according to practical requirements to achieve the object of the scheme of the embodiment. Those of ordinary skill in the art can achieve understanding and implementation without creative work.

From the description of the preceding embodiments, it is apparent to those skilled in the art that the embodiments may be implemented by means of software plus a necessary general-purpose hardware platform or may, of course, be implemented by hardware. Based on this understanding, the preceding technical schemes substantially, or the part contributing to the related art, may be embodied in the form of a software product. The computer software product may be stored in a computer-readable storage medium such as a ROM/RAM, a magnetic disk, or an optical disc and includes several instructions for enabling a computer device (which may be a personal computer, a server, a network device, or the like) to perform the methods described in the embodiments or part of the embodiments.

Finally, it is to be noted that the preceding embodiments are only used to explain the technical schemes of the present disclosure and not to be construed as limitations thereto; though the present disclosure has been described in detail with reference to the preceding embodiments, those of ordinary skill in the art should understand that modifications can be made on the technical schemes in the preceding embodiments or equivalent substitutions can be made on part of the technical features therein; and such modifications or substitutions do not make the essence of the corresponding technical schemes depart from the spirit and scope of the technical schemes in the embodiments of the present disclosure.

What is claimed is:

1. A training method of a brain activity state classification model, comprising:
acquiring pulse sequences of electroencephalography signal samples corresponding to a plurality of training tasks for brain activity state classification; and
inputting the pulse sequences of the electroencephalography signal samples corresponding to the plurality of training tasks into an initial brain activity state classification model separately and training the brain activity state classification model based on a target rule,
wherein training the brain activity state classification model based on the target rule comprises:
in a forward propagation stage in the target rule, updating Hebbian information corresponding to each synapse in the brain activity state classification model according to the pulse sequences corresponding to the plurality of training tasks; and
in a backward propagation stage in the target rule, determining a weight of each synapse in the brain activity state classification model according to the Hebbian information corresponding to each synapse and a backward propagation result;
wherein the Hebbian information is determined based on a co-firing frequency of each synapse, the Hebbian information is used for representing a degree of association between the plurality of training tasks and each synapse, and the brain activity state classification model is established based on a spiking neural network.

2. The training method of claim 1, wherein updating the Hebbian information corresponding to each synapse in the brain activity state classification model according to the pulse sequences corresponding to the plurality of training tasks comprises:
updating the Hebbian information corresponding to each synapse in the brain activity state classification model using the following formulas:
$$\{ \begin{matrix}{H}_{i , j}^{n} = ω {f}_{i , j} + ( 1 - ω ) {H}_{i , j}^{o} \\ {q}_{i , j} = {H}_{i , j}^{n} ( {q}_{i , j} ∈ {Q}_{i} )\end{matrix} ,$$
wherein H_{i,j}^{o} denotes Hebbian information of an i^{th} synapse before a j^{th} task of the plurality of training tasks in the pulse sequences, H_{i,j}ⁿ denotes Hebbian information of the i^{th} synapse after the j^{th} task in the pulse sequences, ω denotes a preset update rate, f_{i,j} denotes a co-firing frequency of the i^{th} synapse in the brain activity state classification model corresponding to the j^{th} task in the pulse sequences, Qᵢ denotes a target list, the Hebbian information of each synapse corresponding to the plurality of training tasks is stored in the target list, and q_{i,j} denotes Hebbian information of the i^{th} synapse corresponding to the j^{th} task stored in the target list.

3. The training method of claim 2, wherein updating the Hebbian information corresponding to each synapse in the brain activity state classification model comprises at least one of the following:
updating the Hebbian information of each synapse based on a co-firing state of each synapse in a single time window; or
updating the Hebbian information of each synapse based on an average firing rate over a plurality of time windows.

4. The training method of claim 3, wherein in the backward propagation stage in the target rule, determining the weight of each synapse in the brain activity state classification model according to the Hebbian information corresponding to each synapse and the backward propagation result comprises:
for any synapse in the brain activity state classification model, in a case where Hebbian information of the synapse is greater than a first threshold, determining that the synapse is associated with a task of the plurality of training tasks and locking a weight of the synapse in the brain activity state classification model; and in a case where the Hebbian information of the synapse is less than or equal to the first threshold, modifying the weight of the synapse according to the backward propagation result.

5. A brain activity state classification method, comprising:
acquiring a pulse sequence corresponding to a target electroencephalography signal; and
inputting the pulse sequence corresponding to the target electroencephalography signal into a brain activity state classification model to obtain a brain activity state classification result, wherein the brain activity state classification model is trained based on the training method of the brain activity state classification model according to claim 1.

6. An electronic device, comprising a memory, a processor, and a computer program stored in the memory and executable by the processor,
wherein the processor, when executing the computer program, performs the following:
acquiring pulse sequences of electroencephalography signal samples corresponding to a plurality of training tasks for brain activity state classification; and
inputting the pulse sequences of the electroencephalography signal samples corresponding to the plurality of training tasks into an initial brain activity state classification model separately and training the brain activity state classification model based on a target rule,
wherein the processor performs training the brain activity state classification model based on the target rule by:
in a forward propagation stage in the target rule, updating Hebbian information corresponding to each synapse in the brain activity state classification model according to the pulse sequences corresponding to the plurality of training tasks; and
in a backward propagation stage in the target rule, determining a weight of each synapse in the brain activity state classification model according to the Hebbian information corresponding to each synapse and a backward propagation result;
wherein the Hebbian information is determined based on a co-firing frequency of each synapse, the Hebbian information is used for representing a degree of association between the plurality of training tasks and each synapse, and the brain activity state classification model is established based on a spiking neural network.

7. The electronic device of claim 6, wherein the processor performs updating the Hebbian information corresponding to each synapse in the brain activity state classification model according to the pulse sequences corresponding to the plurality of training tasks by:
updating the Hebbian information corresponding to each synapse in the brain activity state classification model using the following formulas:
$$\{ \begin{matrix}{H}_{i , j}^{n} = ω {f}_{i , j} + ( 1 - ω ) {H}_{i , j}^{o} \\ {q}_{i , j} = {H}_{i , j}^{n} ( {q}_{i , j} ∈ {Q}_{i} )\end{matrix} ,$$
wherein H_{i,j}^{o} denotes Hebbian information of an i^{th} synapse before a j^{th} task of the plurality of training tasks in the pulse sequences, H_{i,j}ⁿ denotes Hebbian information of the i^{th} synapse after the j^{th} task in the pulse sequences, ω denotes a preset update rate, f_{i,j} denotes a co-firing frequency of the i^{th} synapse in the brain activity state classification model corresponding to the j^{th} task in the pulse sequences, Qⱼ denotes a target list, the Hebbian information of each synapse corresponding to the plurality of training tasks is stored in the target list, and q_{i,j} denotes Hebbian information of the i^{th} synapse corresponding to the j^{th} task stored in the target list.

8. The electronic device of claim 7, wherein the processor performs updating the Hebbian information corresponding to each synapse in the brain activity state classification model by at least one of the following:
updating the Hebbian information of each synapse based on a co-firing state of each synapse in a single time window; or
updating the Hebbian information of each synapse based on an average firing rate over a plurality of time windows.

9. The electronic device of claim 8, wherein in the backward propagation stage in the target rule, the processor performs determining the weight of each synapse in the brain activity state classification model according to the Hebbian information corresponding to each synapse and the backward propagation result by:
for any synapse in the brain activity state classification model, in a case where Hebbian information of the synapse is greater than a first threshold, determining that the synapse is associated with a task of the plurality of training tasks and locking a weight of the synapse in the brain activity state classification model; and in a case where the Hebbian information of the synapse is less than or equal to the first threshold, modifying the weight of the synapse according to the backward propagation result.

10. An electronic device, comprising a memory, a processor, and a computer program stored in the memory and executable by the processor,
wherein the processor, when executing the computer program, performs the following:
acquiring a pulse sequence corresponding to a target electroencephalography signal; and
inputting the pulse sequence corresponding to the target electroencephalography signal into a brain activity state classification model to obtain a brain activity state classification result, wherein the brain activity state classification model is trained based on the training method of the brain activity state classification model according to claim 1.

11. A non-transitory computer-readable storage medium storing a computer program, wherein when executed by a processor, the computer program causes the processor to perform the following:
acquiring pulse sequences of electroencephalography signal samples corresponding to a plurality of training tasks for brain activity state classification; and
inputting the pulse sequences of the electroencephalography signal samples corresponding to the plurality of training tasks into an initial brain activity state classification model separately and training the brain activity state classification model based on a target rule,
wherein the computer program causes the processor to perform training the brain activity state classification model based on the target rule by:
in a forward propagation stage in the target rule, updating Hebbian information corresponding to each synapse in the brain activity state classification model according to the pulse sequences corresponding to the plurality of training tasks; and
in a backward propagation stage in the target rule, determining a weight of each synapse in the brain activity state classification model according to the Hebbian information corresponding to each synapse and a backward propagation result;
wherein the Hebbian information is determined based on a co-firing frequency of each synapse, the Hebbian information is used for representing a degree of association between the plurality of training tasks and each synapse, and the brain activity state classification model is established based on a spiking neural network.

12. The storage medium of claim 11, wherein the computer program causes the processor to perform updating the Hebbian information corresponding to each synapse in the brain activity state classification model according to the pulse sequences corresponding to the plurality of training tasks by:
updating the Hebbian information corresponding to each synapse in the brain activity state classification model using the following formulas:
$$\{ \begin{matrix}{H}_{i , j}^{n} = ω {f}_{i , j} + ( 1 - ω ) {H}_{i , j}^{o} \\ {q}_{i , j} = {H}_{i , j}^{n} ( {q}_{i , j} ∈ {Q}_{i} )\end{matrix} ,$$
wherein H_{i,j}^{o} denotes Hebbian information of an i^{th} synapse before a j^{th} task of the plurality of training tasks in the pulse sequences, H_{i,j}ⁿ denotes Hebbian information of the i^{th} synapse after the j^{th} task in the pulse sequences, ω denotes a preset update rate, f_{i,j} denotes a co-firing frequency of the i^{th} synapse in the brain activity state classification model corresponding to the j^{th} task in the pulse sequences, Qᵢ denotes a target list, the Hebbian information of each synapse corresponding to the plurality of training tasks is stored in the target list, and q_{i,j} denotes Hebbian information of the i^{th} synapse corresponding to the j^{th} task stored in the target list.

13. The storage medium of claim 12, wherein the computer program causes the processor to perform updating the Hebbian information corresponding to each synapse in the brain activity state classification model by at least one of the following:
updating the Hebbian information of each synapse based on a co-firing state of each synapse in a single time window; or
updating the Hebbian information of each synapse based on an average firing rate over a plurality of time windows.

14. The storage medium of claim 13, wherein in the backward propagation stage in the target rule, the computer program causes the processor to perform determining the weight of each synapse in the brain activity state classification model according to the Hebbian information corresponding to each synapse and the backward propagation result by:
for any synapse in the brain activity state classification model, in a case where Hebbian information of the synapse is greater than a first threshold, determining that the synapse is associated with a task of the plurality of training tasks and locking a weight of the synapse in the brain activity state classification model; and in a case where the Hebbian information of the synapse is less than or equal to the first threshold, modifying the weight of the synapse according to the backward propagation result.

15. A non-transitory computer-readable storage medium storing a computer program, wherein when executed by a processor, the computer program causes the processor to perform the following:
acquiring a pulse sequence corresponding to a target electroencephalography signal; and
inputting the pulse sequence corresponding to the target electroencephalography signal into a brain activity state classification model to obtain a brain activity state classification result, wherein the brain activity state classification model is trained based on the training method of the brain activity state classification model according to claim 1.
